(19) United States
(12) Patent Application Publication
(10) Pub. No.: US 2004/0265369 A1
(43) Pub. Date: Dec. 30, 2004

(54) Compounds that inhibit caspase activity for treating glaucoma

(76) Inventor: Stuart A. Lipton, Newton, MA (US)

Correspondence Address: FISH & RICHARDSON PC, 225 FRANKLIN ST, BOSTON, MA, 02110, US

(21) Appl. No.: 10/839,434

(22) Filed: May 5, 2004

Related U.S. Application Data
(63) Continuation of application No. 10/055,417, filed on Jan. 22, 2002, now abandoned.
Application No. 10/055,417 is a continuation of application No. 09/052,826, filed on Mar. 31, 1998, now abandoned.
(60) Provisional application No. 60/042,144, filed on Mar. 31, 1997.

(51) Int. Cl. A61K 48/00; A61K 38/54; A61K 9/127

(52) U.S. Cl. 424/450; 514/44; 514/12

(57) ABSTRACT

Compounds that inhibit caspase activity, particularly those that bind a caspase substrate and protect it, are combined with a vector such as liposomes or an antennapeida peptide to treat glaucoma.

### CROSS REFERENCE TO RELATED APPLICATION

This application is a continuation of U.S. Ser. No. 10/055,417 filed Jan. 22, 2002 which in turn is a continuation of U.S. Ser. No. 09/052,826, filed Mar. 31, 1998 which in turn claimed benefit from provisional application Ser. No. 60/042,144, filed Mar. 31, 1997, each of which is incorporated by reference. WO 98/43621 is also hereby incorporated by reference.

### BACKGROUND OF THE INVENTION

This application is in the general field of treating diseases characterized by apoptosis.

Apoptosis is a programmed cell death which occurs not only in natural development but also in disorders of many tissues incident to certain insults, such as growth factor deprivation and exposure to reactive oxygen species. Apoptosis is implicated, for example in chronic neurodegenerative disorders such as Huntington's disease, amyotrophic lateral sclerosis, Alzheimer's disease, and AIDS dementia, as well as in the penumbra of acute focal cerebral infarcts and after spinal chord injury or other forms of central nervous system trauma. Schwartz and Milligan, Trends in Neurosci. 19:555-562 (1996).

The family of cysteine proteases related to interleukin 1β-converting enzyme (ICE) has been generally found to be essential to apoptosis. Patel et al. FASEB. J. 10:587-797 (1996); Schwartz and Milligan, Trends in Neurosci. 19:555-562 (1996); Troy et al., Proc. Nat'l Acad. Sci. (USA) 93:5635-5640 (1996). The term caspase is now generally used to designate this ICE family of enzymes. Alnemri et al. Cell 87:171 (1996). A conserved cysteine-containing sequence characteristic of caspases is essential for their activity. Patel et al. FASEB. J. 10:587-797 (1996). For all known caspase enzymes, this sequence is QACRG (SEQ ID NO:1). Patel et al. FASEB. J. 10:587-797 (1996). An apoptotic-like neuronal cell death process induced by growth factor deprivation or reactive oxygen species exposure of a neuronal-like cell line (PC12 cells) can be ameliorated by a pseudo-caspase enzyme, a fragment of the natural substrate IQACRG (SEQ ID NO:2) which contains that critical sequence and is believed to complex with and thus protect the natural substrates from degradation by caspases. Troy et al., Proc. Nat'l. Acad. Sci. (USA) 93:5635-5640 (1996).

### SUMMARY OF THE INVENTION

S-nitrosylation (reaction of nitric oxide [NO] species with critical cysteine sulfhydryl groups of a caspase [RS] to form RS-NO) inhibits caspase activity and thereby ameliorates apoptosis. Such inhibition takes place throughout the body, in both neuronal and non-neuronal tissue and in ophthalmological and non-ophthalmological tissues. Accordingly, one aspect of the invention features methods of treating diseases characterized by apoptosis, by administering an S-nitrosylating compound to the patient in an amount effective to reduce caspase activity.

Another aspect of the invention features the use of caspase pseudo-enzymes to treat all apoptotic indications, neurological, ophthalmological, and others. Specifically, apoptotic-like neuronal cell death of cerebrocortical neurons induced by mild excitotoxic injury [see, Bonfoco et al. Proc. Nat'l Acad. Sci. (USA) 92:7162-7166 (1995)] can be ameliorated by caspase substrate binding agent—peptides containing the sequence QACRG (SEQ ID NO:1), particularly those containing IQACRG (SEQ ID NO:2) and most particularly, IQACRG (SEQ ID NO:2) itself. These peptides may be linked to an antennapedia sequence (see Troy et al., cited above, which is hereby incorporated by reference) or they may be incorporated into liposomes to enhance transport across the blood-brain barrier and/or entry into neurons.

Finally the two approaches (nitrosylating therapies and caspase substrate binding agent) may be combined to treat apoptotic indications.

Other features and advantages will be apparent from the following description of the Preferred Embodiments and from the claims.

### BRIEF DESCRIPTION OF THE DRAWINGS

FIG. 1 is a bar graph depicting inhibition of caspase-induced opoptosis by endogenous NO (See Example 1).

FIG. 2 is a bar graph depicting the results of an experiment (Example 2) in which V-ICEᵢₙₕ decreases apoptosis induced by N-methyl-D-aspartate (NMDA).

### DESCRIPTION OF THE PREFERRED EMBODIMENTS

Among the non-neuronal medical indications that can be treated according to the invention are: autoimmune diseases, including diseases of lymphocytes, systemic lupus erythematosus (SLE), synovial cells of rheumatoid arthritis (RA), fibroblasts (scleroderma), defective hematopoiesis, atherosclerosis, gastrointestinal diseases associated with cell death, including hepatobiliary disease, cell-mediated cytotoxicity, drug and chemical toxicity, carcinogenesis, viral disease, T-cell depletion associated with AIDS, oxidative stress, glomerulonephritis, cystic renal disease, renal tubular injury, atherosclerosis, myocardial ischemia or infarction, diabetic nephropathies, Chagas' disease polycystic kidney disease, glomerulonephritis, hypocellular end-stage kidney disease, kidney disease associated with diabetes mellitus, Sjögren's syndrome, fulminant hepatitis (hepatitis B and C), red cell pathology; polycythemia, thalassemia, deficiencies in folate, vitamin B12, iron, glucose-6-phosphate dehydrogenase abnormalities, bone marrow failure, myelodysplasia, and chronic inflammatory disease.

Neuronal medical indications include Parkinson's disease, Alzheimer's disease, Amyotrophic lateral sclerosis, autoimmune inflammation of the nervous system, multiple sclerosis, demyelinating diseases, autoimmune encephalomyelitis, status epilepticus and other seizure disorders, neurological mechanical trauma, hypoxia hypoglycemia, and ischemia, optic neuropathies, glaucoma, AIDS dementia, stroke, neuropathic pain, Huntington's disease, metabolic disorders (including homocyst(e)inemia) Tourette's syndrome, and withdrawal from drug addiction, drug tolerance or drug dependency.

The S-nitrosylating therapeutics that can be used to effect treatment according to the invention include any compound which produces a sufficient amount of NO (most probably a related redox species such as an NO⁺ equivalent or NO⁻ donor) upon administration to a mammal to decrease apoptotic damage or injury. For convenience, I have also used the less precise term “NO-generating compound” to include compounds that produce the above described NO-related redox species (e.g., RS-NO, an NO⁺ equivalent, or NO⁻) or a physiologically acceptable salt thereof.

Verification that a particular compound nitrosylates a caspase can be accomplished by the experiments provided below.

The two preferred compounds (nitroglycerin and sodium nitroprusside) provide the advantage of a proven record of safe human administration (i.e., for treatment for cardiovascular disorders). Other nitroso-compounds that can be used in the method of the invention include: isosorbide dinitrate (isordil); S-nitroso captopril (SNOCAP); Serum albumin coupled to nitric oxide (“SA-NO”); Cathepsin coupled to nitric oxide (cathepsin—NO); tissue plasminogen activator coupled to NO (tPA-NO); SIN-1 (or molsidomine) cation-nitrosyl complexes, including Fe²⁺-nitrosyl complexes; Nicorandil; S-nitrosoglutathione; NO coupled to an adamantine derivative, such as memantine (see U.S. Pat. No. 5,614,650 hereby incorporated by reference); S-nitrosothiols including S-nitrosocysteine; quinones, including pyrroloquinoline quinone (PQQ), ester derivatives of PQQ, or ubiquinone; sydnonimines or NONOates having the formula

X—[N (O) NO]⁻

where X is any nucleophile including an amine; and agents which generate an oxidizing cascade similar to that generated by NO such as α-lipoic acid (thioctic acid and its enantiomers); dihydrolipoate; glutathione; ascorbate; or vitamin E. Alternatively, the NO donor can be a nitroxyl (NO⁻) generator such as Piloty's acid, Angeli's salt (Oxi-NO), or sulfi-NO. See generally the list of NO compounds described in Chapter 7 of Feelisch and Stamler, Methods in Nitric Oxide Research, Wiley and Sons, Chichester, UK, (1996), pp 71-115, which is hereby incorporated by reference. Without wishing to be bound to a specific theory, the NO group in various redox forms can be transferred or react with the critical cysteine at the active site of caspases to decrease enzymatic function and thus provide protection against apoptosis.

Any of the above described nitroso-compounds may be combined with other redox compounds that facilitate production and maintenance of NO. For example, direct NO-generators can be combined with pyroloquinoline quinone (PQQ) (see U.S. Pat. No. 5,091,391), or PQQ's derivative esters, or other quinones such as ubiquinone.

The ability of NO to be transported to and cross cell membranes facilitates therapies according to the invention.

My earlier U.S. patent U.S. Pat. No. 5,455,279 discloses that it is possible to build tolerance to undesired cardiovascular side effects of NO compounds (e.g., hypotension), without losing the desired protective effect. Accordingly, nitroso compounds capable of protecting against apoptosis can be administered continuously over an extended period with gradually escalating dosage, beginning at a dosage level which does not substantially reduce the patient's blood pressure, and, later, increasing gradually to higher dosage levels desirable for achieving the anti-apoptotic effect. The later dosage level is high enough to substantially reduce a naive patient's blood pressure, but, due to the tolerance that has been achieved in the patient, the compound's blood-pressure lowering effect is reduced to tolerable levels.

An alternative way to offset the hypotensive effects of NO donors such as nitroglycerin is to co-administer with the NO-donating compounds, agents such as phenylephrine, dopamine, or yohimbine. See, e.g., Ma et al. Cardiovasc. Pharmacol. 20: 826-836 (1992). These agents may be given parenterally (e.g. IV) or orally depending on the drug.

Nitroglycerin may be administered by transdermal patch as described in detail in my U.S. Pat. No. 5,455,279, referenced above. Alternatively, a long-lasting nitrate formulation, such as isosorbide dinitrate SR tablets which are usually administered every 8-12 hours, are administered more frequently (e.g., every 4 hours) to induce cardiovascular tolerance but preserve their effect on nitrosylation of caspases. It is also useful to administer superoxide dismutase (SOD), catalase, or both, to limit toxicity by decreasing the formation of peroxynitrite from the reaction of NO with superoxide anion (O₂^{·-}).

The compound may be included in a pharmaceutical preparation, using a pharmaceutical carrier (e.g., physiological saline); the exact formulation of the therapeutic mixture depends upon the route of administration. Preferably, the compound is administered orally or intravenously, but it may also be administered sublingually, by nasal spray, by transdermal patch, subcutaneously, intraventricularly, intravitreally, or by ointment. The preferred compounds, nitroglycerin or their derivatives (including all those preparations commercially available, e.g., those listed in the Physician's Desk Reference (1997) under coronary vasodilators or under nitroglycerin or nitroglycerin intravenous and including isosorbide mononitrate, isosorbide dinitrate, nitroglycerin sublingual, Minitran, NT-1, Niotrocor, Nitroderm, Nitrodisc, Nitro-dur, Nitro-Dur II, Nitrofilm, Nitrogard, Nitroglin, Nitropen, Tridil, and 6-chloro-2-pyridylmethyl nitrate) are administered at 0.01 mg-60 gm/day, in divided doses. Sodium nitroprusside—Na₂[Fe(CN)₅NO]−2H₂O (from Elkins-Sinn, Inc., Cherry Hill N.J.), Nipride (from Roche, Nutley, N.J.), or other preparations—are administered intravenously at 0.5-10 μg/min.

Compounds determined to be effective protective agents by the assays described herein are administered as above at a dosage suitable to reduce cellular damage. Generally, such compounds are administered in dosages ranging from 0.01 mg-60 gm/day, more preferably in dosage of 0.1-5 mg/day.

Those skilled in the art will understand that there are other factors which aid in determining optimum dosage. For example, for NO-conjugated drugs, the dosage used for the unconjugated drug (e.g. tPA a dosage of 0.35-1.08 mg/kg and generally≦0.9 mg/kg) is predictive of useful NO-conjugate dosage. Dosages may be divided. It is desirable to maintain levels of NO or related redox species in the brain of 1 nM to 500 μM. Treatment may be repeated as necessary.

Regarding neuronal therapies, polyethylene glycol (PEG) is used to enhance absorption into the central nervous system (CNS) and efficacy of SOD and/or catalase. An SOD mimic, the protein-bound polysaccharide of Coriolus versicolor QUEL, termed “PS-K”, may also be effective by parenteral or oral routes of administration, especially with PEG to enhance CNS absorption, and such mimics may be substituted for SOD in this aspect of the invention. See Kariya et al., Mol. Biother. 4:40-46 (1992); and Liu et al., (1989) Am. J. Physiol. 256:589-593.”

### EXAMPLES

### Example 1

We have shown that S-nitrosylation of caspases [e.g., CPP32 (caspase -3, Alnemri et al.) and ICE (caspase-1)] inhibit their ability to cleave the substrate PARP [poly(ADP-ribose)polymerase]. Fluorogenic assays of caspase activity in neuronal and other cellular cultures revealed that S-nitrosylation by either exogenous or endogenous NO species inhibited enzyme activity and therefore prevented apoptosis.

Nitrosylation of the critical cysteine in caspases (which is present in the peptide ICARG) (SEQ ID NO:3) can be verified by the Saville reaction, well known to those skilled the art. Feelish and Stamler, cited above, Ch. 36, p. 527.

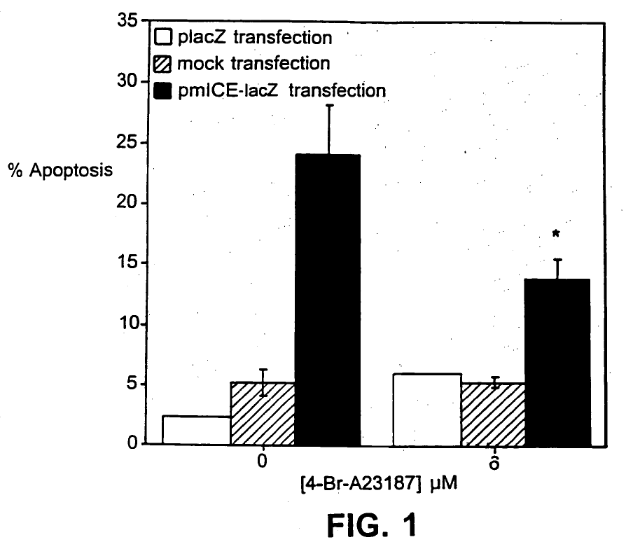

In cell toxicity experiments we demonstrate inhibition of caspase-induced apoptosis by endogenous NO in HEK-293-nNOS cells. HEK-293 cells [Bredt et al., Nature 351:714-719 (199 )} overexpressing nNOS were transiently transfected with mICE-lacZ (containing the caspase-1 construct [Miura et al., Cell 75:653-660 (1993)] or control placZ using the calcium phosphate precipitation method. Following transfection, cells were incubated in absence (0 μM) or presence of 6 μM 4-Br-A23187 for 48 h. Cells were then permeabilized, fixed, and stained with propidium iodide. Apoptotic nuclei were counted in ≧12 fields and results expressed as a percentage of total nuclei. The results are shown in FIG. 1. Values are the mean±SEM for n≧3 from at least two experiments. A Fisher's protected least significance difference post-hoc test indicated a highly significant decrease in apoptosis of HEK-293-nNOS cells after caspase-1 transfection and 4-Br-A23187 exposure to increase Ca²⁺ and thus activate the nNOS to produce NO (p≦0.007).

### Example 2

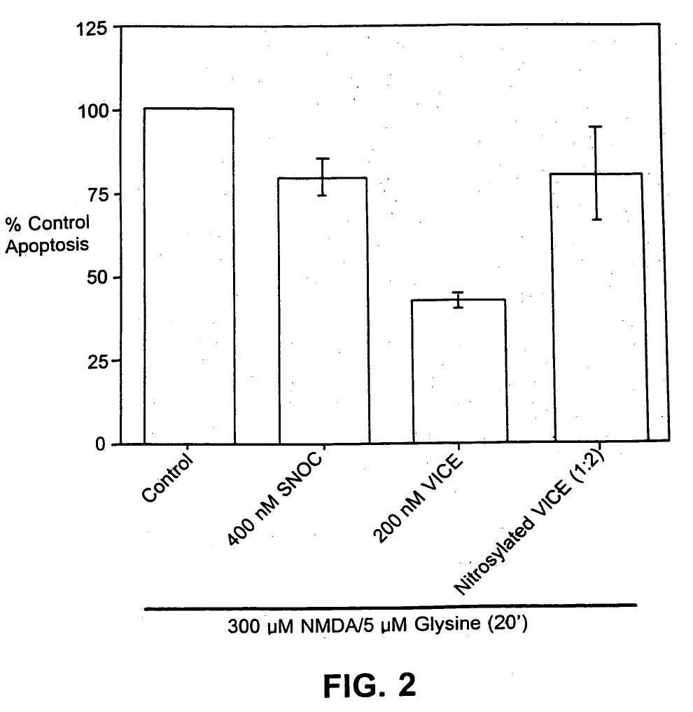

FIG. 2 depicts the results of one specific experiment in which the pseudo-caspase enzyme IQACRG (“ICEᵢₙₕ”) demonstrably decreases the apoptosis induced by the excitotoxin N-methyl-D-aspartate (NMDA) plus glycine (an NMDA receptor co-agonist.) Note that ICEᵢₙₕ's entry into cells is facilitated by coupling the antennapedia peptide (a signal sequence allowing translocation across cell membranes, the conjugate being termed V-ICEᵢₙₕ). Note also that the NMDA receptor is a subtype of glutamate receptor, which, when overexcited, causes neuronal damage. The reduction in NMDA-induced (300 μM NMDA/5 μM glycine) neuronal apoptosis effected by 200 nM VICE is significant.

These findings support my conclusion that S-nitrosylation of caspase inhibits apoptosis. The pseudo-enzyme IQACRG (SEQ ID NO:2) containing the caspase active site also prevents apoptosis. The combination of the two is synergistic.

What is claimed is:

1. A method of treating glaucoma in a patient in need thereof, the method comprising administration to said patient of a therapeutic composition comprising a caspase substrate binding agent and a transport-enhancing vector.

2. The method of claim 1 in which the transport enhancing vector comprises liposomes.

3. The method of claim 1 in which the caspase substrate binding agent is a peptide that comprises the sequence QACRG.

4. The method of claim 1 in which the caspase substrate binding agent is apeptide that comprises the sequence IQACRG.

5. The method of claim 1 in which the caspase substrate binding agent is a peptide having the sequence IQACRG.

6. The method of claim 1, claim 3, claim 4, or claim 5 in which the vector comprises an antennapeida peptide.

7. The method of claim 1 in which the composition is administered intravitreally.

8. A method of treating glaucoma in a patient in need thereof, the method comprising administration to said patient of a therapeutic composition a caspase activity inhibiting agent and a transport-enhancing vector.


SEQUENCE LISTING (3 sequences)

SEQ ID NO: 1 - protein, 5 aa, Homo sapiens
QACRG

SEQ ID NO: 2 - protein, 6 aa, Homo sapiens
IQACRG

SEQ ID NO: 3 - protein, 5 aa, Homo sapiens
ICARG